(19) United States
(12) Patent Application Publication
(10) Pub. No.: US 2006/0142288 A1
(43) Pub. Date: Jun. 29, 2006

(54) Combinations and methods for headaches

(76) Inventor: Stephen Peroutka, (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/294,345

(22) Filed: Dec. 6, 2005

Related U.S. Application Data
(60) Provisional application No. 60/633,430, filed on Dec. 7, 2004.

(51) Int. Cl. A61K 31/537; A61K 31/192; A61K 31/137

(52) U.S. Cl. 514/238.8; 514/569; 514/570; 514/651

(57) ABSTRACT

The present invention provides pharmaceutical agents for preventing headaches, especially headaches associated with migraine, tension headaches, and chronic daily headaches. The prophylactic treatments in accordance with the present invention can reduce the frequency of headaches, such as migraine attacks, as well as reducing their severity and duration when they do occur. The pharmaceutical agents can comprise a combination of a cyclooxygenase-3 inhibitor and beta-adrenergic receptor antagonist in amounts which are effective to achieve the desired purpose of preventing headaches. In addition, the present invention provides compositions and methods for treating headaches, comprising administering a cyclooxygenase-3 inhibitor.

This application claims the benefit of the filing date of U.S. Provisional Application Ser. No. 60/633,430 filed Dec. 7, 2004 which is incorporated by reference herein.

### DESCRIPTION OF THE INVENTION

The present invention provides pharmaceutical agents for preventing headaches, especially headaches associated with migraine, tension headaches, and chronic daily headaches. The prophylactic treatments in accordance with the present invention can reduce the frequency of headaches, such as migraine attacks, as well as reducing their severity and duration when they do occur. The pharmaceutical agents can comprise a combination of a cyclooxygenase-3 inhibitor and beta-adrenergic receptor antagonist in amounts which are effective to achieve the desired purpose of preventing headaches. In addition, the present invention provides compositions and methods for treating headaches, comprising administering a cyclooxygenase-3 inhibitor, including selective inhibitors of it.

Cyclooxygenases (COX) catalyze the first and rate-limiting step of prostaglandin synthesis from arachidonic acid in cellular membranes. They are present in a wide variety of tissues in the periphery and central nervous system. Several different cyclooxygenase isoenzymes have been identified, including COX-1, COX-2, and COX-3.

COX-1 is constitutively expressed in nearly all tissues throughout the body and plays a major role in many physiological functions such as protection of the gastric mucosa and platelet aggregability. In contrast, expression of COX-2 is highly restricted, but is dramatically up-regulated during inflammation. A third distinct COX isozyme, called COX-3, has also been described (Chandrasekharan et al., Proc. Natl. Acad. Sci., 99(21):13926-31, 2002). COX-3 is derived from the COX-1 gene, but retains intron 1 in its mRNA. For this reason, it is also referred to as referred to as COX-1 splice variant 1 or COX-1V₁. Translation of this alternatively spliced RNA results in a polypeptide with an N-terminal extension coded for by intron-1 and the retained signal peptide. COX-3 is expressed, e.g., in sensory neurons.

Comparison of canine COX-3 activity with murine COX-1 and COX-2 demonstrated that analgesic/antipyretic drugs such as acetaminophen, phenacetin, antipyrine, and dipyrone selectively inhibit this enzyme. In addition, drugs that block COX-1 can also block COX-3. Thus, COX-3 is also potently inhibited by some NSAIDs such as ibuprofen and diclofenac (Chandrasekharan et al., 2002).

COX inhibitors have been used to both treat and prevent headaches. For example, a six year randomized trial conducted among 5139 healthy male British physicians reported that 500 mg acetylsalicyclic acid (ASA: aspirin) daily reduced the incidence of migraine by 30% (Peto et al., 1988, Br. Med. J. (Clin. Res. Ed) 296: 313-316). In a small prospective double-blind pilot study of higher doses of ASA (i.e., 650 mg/day), a reduction of more than 50% in headache frequency was reported in 9 of 12 migraine patients (O'Neill B P and Mann J D, 1978, Lancet 2: 1179-1181).

In a double blind cross-over study, overall migraine frequency was reduced significantly (p<0.001) with ASA (500 mg TID) (Grotemeyer K H et al., 1990, Headache 30: 639-641). However, a reduction of attacks >50% was seen with ASA in only 3 of 22 subjects (14%). The authors concluded that even though ASA was of statistically significant efficacy in migraine prophylaxis, it clearly is not the drug of first choice in migraine since, in the same study, a reduction of attacks >50% was seen in 14 of 21 subjects (67%) taking metoprolol (Grotemeyer et al., 1990). In another small (n=18) double-blind crossover study, ASA (13.5 mg/kg/day or ˜1 g per day) was reported to significantly decrease the frequency, severity and duration of migraine attacks (Baldrati A. et al., 1983, Acta Neurol. Scand 67: 181-186).

Similar results have been reported for other COX inhibitors. Several publications have focused on COX-2 activity. For example, U.S. Pat. No. 6,384,034 describes a method of preventing migraine in a mammalian patient which comprises administering a COX-2 selective inhibiting compound and a 5HT-1B/1D agonist. U.S. Patent Application 20040186155 (filed Jan. 29, 2004) describes a method for treating or preventing migraine in a mammalian patient comprising concomitantly or sequentially administering a cyclooxygenase-2 selective inhibitor and a beta adrenergic receptor blocking agent in amounts that are effective for treating or preventing migraine. U.S. Patent Application 20040151792 provides compositions that inhibit expression of COX-2 for treating pathological conditions associated with inflammatory response, and identified migraine in a long list of disease.

In contrast to these reports, the present invention provides methods for preventing headaches utilizing a cyclooxygenase-3 inhibitor. The phrase “cyclooxygenase inhibitor” indicates that the agent inhibits or blocks the enzyme activity of the cyclooxygenase enzyme. Such an inhibitor can block cyclooxygenase-1 and/or -2 activity as well, but blockade of these is not necessary to achieve therapeutic efficacy in preventing headaches. The ability to inhibit cyclooxygenase activity can be measured routinely. For example, COX activity can be measured by the production of a prostaglandin, such as PGE₂, in either cell-based or cell-free assays. Cyclooxygenase assays are also described in, e.g., Brideau et al., Inflamm Res., 45: 68-74 (1996); U.S. Pat. No. 6,825,185; U.S. Pat. No. 6,812,346; U.S. Pat. No. 6,649,636; U.S. Pat. Application 20040235017 (COX-3 assay). COX assays are available as commercial kits, as well.

Cyclooxygenase inhibitors can be selective or non-selective. Selectivity refers to the ability to block one isoenzyme form more potently than another form. A cyclooxygenase-3 selective inhibitor is therefore an agent that is more potent at blocking COX-3 enzyme activity, than either COX-1 or -2. The amount of selectivity can vary, e.g., a COX-3 selective inhibitor can be from about 2-fold, 5-fold, 10-fold, 50-fold, 100-fold, 1000-fold, etc., more potent at inhibiting COX-3 activity as compared to COX-1 or COX-2 activity. A non-selective agent is an inhibitor that is substantially equipotent on all three isoforms, without substantially no preference for either form. Table 1 from Chandrasekharan et al. provides examples of COX inhibitors and their IC₅₀ for the three different COX isoenzymes.

A COX-3 inhibitor can have substantially no COX-1 and/or COX-2 inhibitory activity. For example, when a COX-3 inhibitor is administered to a subject, if the amounts in the body are insufficient to achieve a therapeutic effect via COX-2, this would be considered to have substantially no COX-2 inhibitory activity.

Any agent that possesses COX-3 inhibitory activity can be used to prophylactically manage headaches. Activity can measured as described above. Non-steroidal anti-inflammatory drugs (NSAIDs) are a major class of pharmacological agents which can act as cyclooxygenase-3 inhibitors. Numerous NSAIDs have been approved for the acute treatment of migraine (e.g., acetaminophen, acetylsalicylate acid, diclofenac, ibuprofen, rofecoxib, tolfenamic acid). However, no NSAID has ever been approved for use as a migraine prophylactic agent. Useful NSAISs that can have COX-3 activity, include, but are not limited to, e.g., Salicylic acid derivatives, such as aspirin, sodium salicylate, choline magnesium trisalicylate, salsalate, diflunisal, salicylsalicylic acid, sulfasalazine, and olsalazine; Para-aminophenol derivatives, such as acetaminophen; Indole and indene acetic acids, such as indomethacin, sulindac, and etodolac; Heteroaryl acetic acids, such as tolmetin, diclofenac, and ketorolac; Arylpropionic acids, such as ibuprofen, naproxen, flurbiprofen, ketoprofen, fenoprofen, oxaprozin; Anthranilic acids (fenamates), such as mefenamic acid, and meclofenamic acid; Enolic acids, such as xicams (piroxicam, tenoxicam), and pyrazolidinediones (phenylbutazone, oxyphenthatrazone); Alkanones, such as nabumetone. Specific examples include: aspirin, fenoprofen (Nalflon®), flurbiprofen (Ansaid®), flufenamic acid, ketoprofen (Orudis®), mefenamic acid (Ponstel®), meloxicam (Mobic®), babumetone (Relafen®), naproxen (Aleve®; Naprelan®, Oxaprozin (Daypro®), Prioxicam (Feldene®), tolfenamic acid, derivatives, and pharmacologically-active salts thereof. Examples of COX-3 selective inhibitors include, e.g., acetaminophen, phenacetin, dipyrone, diclofenac, aspirin, ibuprofen, derivatives thereof, and pharmacologically-active salts thereof.

The present invention also provides methods of preventing headaches in a subject in need thereof, comprising, administering to said subject a beta-adrenergic receptor antagonist and cyclooxygenase-3 inhibitor in amounts which are effective to prevent said headaches, and pharmaceutical combinations that comprise, or consist essentially of, effective amounts of a beta-adrenergic receptor antagonist and cyclooxygenase-3 inhibitor.

A beta-adrenergic receptor antagonist (beta-blocker) is any compound that blocks the biological activity of the beta-adrenergic receptor, e.g., by binding to the receptor without eliciting the biological response normally stimulated by the receptor agonist (epinephrine or norepinephrine). Receptor antagonism can be competitive, where the antagonist competes directly with the agonist at the receptor's ligand binding site, or it can be non-competitive. There are several different subtypes of the beta-adrenergic receptor, including, beta-1, beta-2, and beta-3. All subtypes have been reported to be coupled to the stimulation of adenylyl cyclase activity.

Both beta-1-adrenergic selective and non-selective beta-adrenergic receptor antagonists can be utilized in combination with a COX-3 inhibitor. Preferred antagonists are those that lack intrinsic sympathormimetic activity (“ISA”) (i.e., partial agonist activity) (Fanchamps A., 1985, Headache 25: 61-62). Examples of antagonists that lack ISA and which have previously been shown to be effective in prophylactic treatment of migraine, include, atenolol (Tenormin®), metoprolol (Lopressor®; Toprol-XL®), nadolol (Corgard®), Bisoprolol (Zebeta®), timolol (Blocadren®), and derivatives thereof, and pharmacologically-active salts thereof.

Examples of non-subtype selective beta-adrenergic antagonists include, but are not limited to, e.g., propranolol (Inderal®, Inderal-LA®), nadolol (Corgard®), timolol (Blocadren®), derivatives thereof, and pharmacologically-active salts thereof.

Examples of selective beta-1-adrenergic receptor antagonists include, but are not limited to, e.g., metoprolol (Lopressor®; Durules®, Toprol-XL®), esmolol, bisoprolol (Zebeta®), Nebivolol (Mylan®), Atenolol (Tenormin®), derivatives thereof, and pharmacologically-active salts thereof.

Headaches that can be treated or prevented in accordance with the present invention include, e.g., migraine without aura, migraine with aura, tension-type headache, premenstrual headache, chronic daily headache, etc. Migraine without aura can be associated with, e.g., hemicranial- or bilateral pain, pulsating head pain, steady nonpulsatile head pain, nausea, vomiting, photophobia, phonophobia, and osmophobia. Migraine with aura has similar symptoms, but subjects also experience aura. Subjects with tension-type headaches can experience, e.g., bilateral, occipital, or frontal head pain, aching, tight, and squeezing head pain, and nausea. For more information on headaches, see, e.g., Olesen, Cephalalgia, Vol. 8, Supplement 7, 1988. In addition to reducing the frequency, severity and duration of headaches, methods of the present invention can also reduce, eliminate, decrease, etc., the frequency, severity and duration of one or more the aforementioned symptoms, or any other symptom that is associated with a headache or migraine attack.

By the term “preventing,” it is meant that the COX-3 in combination with a beta-adrenerig receptor antagonist reduces the frequency, severity, intensity, and/or duration of a headache when administered prior to the headache's onset. An amount of the active agent that is effective in preventing the headache is any amount that reduces the frequency, severity, intensity, and/or duration of headaches in a subject being treated. The specific dose level and frequency of dosage may vary, and can depend upon a variety of factors, including the activity of the specific active agents, their metabolic stability and length of action, rate of excretion, mode and time of administration, and the age, body weight, general health, gender, diet, and severity, intensity, and frequency of the onset of the headache, of the particular condition of the subject undergoing therapy. An active agent in accordance with the present invention can be immediately effective in achieving prophylaxis, or can reach its maximal effect after multiple, regular doses, e.g., one or more doses a day for a week, two weeks, a month, three months, etc.

An agent's efficacy in reducing the frequency of headache can be determined routinely, e.g., by determining how many headaches occur in given time period, before and after treatment is initiated, or by determining the interval between headache occurrences.

The term “treating” is used conventionally, e.g., the management or care of a subject for the purpose of combating, alleviating, reducing, relieving, improving, etc., a headache. Any amount of improvement in the perception of headache pain and discomfort is considered useful, and the clinical efficacy of such treatment can be assessed using any of the scales conventionally utilized by a healthcare practitioner, e.g., no pain, mild pain, moderate pain, or severe pain. Headache pain can be measured at the onset, and then at 0.5, 1, 1.5, 2 hours, and longer intervals.

The combination of the cyclooxyenase-3 inhibitor and beta-adrenergic receptor can be administered at any times and in any effective form. For example, the compounds can be administered simultaneously, e.g., as a single composition or dosage unit (e.g., a pill or liquid containing both compositions), or they can be administered as separate compositions, but at the same time (e.g., where a subject is administered a composition containing the COX-3 inhibitor, and a second composition containing the beta-adrenergic blocker). The active agents can also be administered sequentially at different times. In certain embodiments of the present invention, the agents can be administered simultaneously and once every 24-hours, e.g., just before a subject is going to sleep for a night (bedtime). Agents can be formulated conventionally to achieve the desired rates of release over extended period of times, e.g., 12-hours, 24-hours. This can be achieved by using agents and/or their derivatives which have suitable metabolic half-lives, and/or by using controlled release formulations.

The phrase “controlled release” indicates that the release of the active ingredient is regulated or modulated to achieve a desired rate of delivery into the systemic circulation. A controlled release formulation can be pulsed, delayed, extended, slow, steady, immediate, rapid, fast, etc. It can comprise one or more release formulations, e.g. extended- and immediate-release components. For example, an oral controlled release formulation can comprise a plurality of components positioned in any suitable arrangement, e.g., comprising a “free” drug in a rapidly soluble polymer film on the outside of the dosage unit to achieve an immediate therapeutic effect, and an extended release delivery system in the core of the unit to produce steady state concentrations of active agents. A formulation can be a composition of matter, a device, a patch, multi-layered or multi-configured products, etc. An extended release composition is one in which the active ingredient is not released immediately in its active form, but is slowly and controllably discharged from the dosage unit. The kinetics of the extended release are influenced by the choice of the delivery system, amount of the active ingredient, dissolution rate of the drug, compartment in which release occurs (e.g., with oral delivery systems, this is the gastrointestinal tract), absorption of drug from the site of release into the systemic circulation, drug distribution from the systemic circulation, etc. An immediate release formulation can be used to deliver the equivalent of a “bolus” to the body, releasing the active form of the drug directly into the targeted physiological compartment (e.g. the GI tract) to achieve rapid systemic availability.

Any effective amounts of the agents can be used. An effective amount is the amount of an agent (or combination) that is successful in achieving the desired purpose. The combination of agents can be synergistic, i.e., where the joint action of the agents is such that the combined effect is greater than the algebraic sum of their individual effects. The specific dose level and frequency of dosage may vary, and can depend upon a variety of factors, including the activity of the specific active agents, their metabolic stability and length of action, rate of excretion, mode and time of administration, and the age, body weight, general health, gender, diet, and severity, intensity, and frequency of the onset of the headache, of the particular condition of the subject undergoing therapy.

The present invention also relates to a pharmaceutical combination comprising, or consisting essentially of, a cyclooxygenase-3 inhibitor and a beta-adrenergic receptor antagonist. The combination can be in the form of a package or packet, e.g., a unit comprising individually sealed compartments and containing one or more prescribed solid oral dosage forms in each compartment (such as blister packaging). For example, a monthly dosage unit can have pills arranged in separate compartments, where there are two compartments for each day of the month—one for a cyclooxygenase-3 inhibitor and a second for a beta-adrenergic receptor antagonist. The combination can also be a composition containing effective amounts of the inhibitor and antagonist, where a single dosage unit comprises both agents.

The present invention also relates to methods of making pharmaceutical preparations for headaches which comprise a step of determining that an agent is a cyclooxygenase-3 inhibitor, and preparing a composition that comprises the inhibitor. The determining step indicates that the agent is actively identified as possessing cyclooxygenase-3 inhibitory activity. For example, the agent can be selected as a therapeutic or prophylactic agent because of its COX-3 activity. The determining step can be performed only once in the preparative process, where at least at one point, the agent is determined as having the desired activity. Thus, a COX assay does not have to be performed on every batch. The determination can be through laboratory experiment where the activity is routinely assayed using any suitable measure for COX activity, or it can be based on data from other sources, e.g., publications and other written materials. Methods for formulating pharmaceutical preparations are well known in the art.

Any combination of a COX-3 inhibitor and a beta-adrenergic receptor antagonist can be used, e.g., selecting from any of the compounds expressly mentioned herein. Useful combinations included, e.g., naproxen and metoprolol; naproxen and propranolol; meloxicam and metoprolol; meloxicam and propranolol. These can be formulated such that each agent is long-acting, requiring administration once a day (e.g., at bedtime), or twice a day.

Active agents in accordance with the present invention can be administered in any form by any effective route, including, e.g., oral, parenteral, enteral, intraperitoneal, topical, transdermal (e.g., using any standard patch), ophthalmic, nasally, local, non-oral, such as aerosal, spray, inhalation, subcutaneous, intravenous, intramuscular, buccal, sublingual, rectal, vaginal, intra-arterial, and intrathecal, etc. It can be administered alone, or in combination with any ingredient(s), active or inactive.

In addition to the substances already mentioned, active agents can be further combined with any other suitable additive or pharmaceutically acceptable carrier. Such additives include any of the substances already mentioned, as well as any of those used conventionally, such as those described in Remington: The Science and Practice of Pharmacy (Gennaro and Gennaro, eds, 20^{th} edition, Lippincott Williams & Wilkins, 2000); Theory and Practice of Industrial Pharmacy (Lachman et al., eds., 3^{rd} edition, Lippincott Williams & Wilkins, 1986); Encyclopedia of Pharmaceutical Technology (Swarbrick and Boylan, eds., 2^{nd} edition, Marcel Dekker, 2002).

These are generally referred to herein as “pharmaceutically acceptable carriers” to indicate they are combined with the active drug and can be administered safely to a subject for therapeutic or prophylactic purposes. These include, but are not limited to, antioxidants, preservatives, dyes, tablet-coating compositions, plasticizers, inert carriers, excipients, polymers, coating materials, osmotic barriers, devices and agents which slow or retard solubility, etc.

The active agent of this invention can be in any suitable form, without limitation. Forms suitable for oral use, include, but are not limited to, tablets, troches, lozenges, aqueous or oily suspensions, dispersible powders or granules, emulsions, hard or soft capsules, solutions, syrups and elixirs. Compositions intended for oral use may be prepared according to any method known to the art for the manufacture of pharmaceutical compositions. Suitable pharmaceutically acceptable carriers include but are not limited to water, salt solutions, alcohols, gum arabic, vegetable oils, benzyl alcohols, gelatin, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose and the like. Other additives include, e.g., antioxidants and preservatives, coloring, flavoring and diluting agents, emulsifying and suspending agents, such as acacia, agar, alginic acid, sodium alginate, bentonite, carbomer, carrageenan, carboxymethylcellulose, cellulose, cholesterol, gelatin, hydroxyethyl cellulose, hydroxppropyl cellulose, hydroxypropyl methylcellulose, methylcellulose, octoxynol 9, oleyl alcohol, povidone, propylene glycol monostearate, sodium lauryl sulfate, sorbitan esters, stearyl alcohol, tragacanth, xanthan gum, and derivatives thereof, solvents, and miscellaneous ingredients such as microcrystalline cellulose, citric acid, dextrin, dextrose, liquid glucose, lactic acid, lactose, magnesium chloride, potassium metaphosphate, starch, and the like.

The active agent or the novel composition of this invention may be in a form suitable for oral use, for example, tablets, troches, lozenges, aqueous or oily suspensions, dispersible powders or granules, emulsions, hard or soft capsules, solutions, syrups and elixirs. Compositions intended for oral use may be prepared according to any method known to the art for the manufacture of pharmaceutical compositions and typically such compositions contain one or more agents selected from the group consisting of sweetening agents, flavoring agents, coloring agents and preservatives in order to provide pharmaceutically elegant and palatable preparations. These excipients may be for example, diluents such as lactose, calcium carbonate, sodium carbonate, calcium phosphate or sodium phosphate; granulating and disintegrating agents, for example, corn starch or alginic acid; binding agents, for example starch, gelatin or acacia, and lubricating agents, for example, magnesium stearate, stearic acid or talc.

The tablets may be uncoated or they may be coated. Coating can be included to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pats. Nos. 4,256,108; 4,166,452; and 4,265,874 to form osmotic therapeutic tablets for control release. Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or miscible solvents such as propylene glycol, PEGs and ethanol, or an oil medium, for example peanut oil, liquid paraffin or olive oil.

Aqueous suspensions contain the active material in admixture with excipients suitable for the manufacture of aqueous suspensions. Such excipients are suspending agents, for example sodium carboxymethylcellulose, methylcellulose, hydroxy-propylmethycellulose, sodium alginate, polyvinyl-pyrrolidone, tragacanth and acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl or n-propyl p-hydroxybenzoate, one or more colouring agents, one or more flavouring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

The individual agents or the pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxy-ethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain demulcents, preservatives, flavorants and coloring agents.

The pharmaceutical compositions may be in the form of a sterile injectable aqueous or oleagenous suspension. This suspension may be formulated according to the known art using those suitable dispersing or wetting agents and suspending agents which have been mentioned above.

Injectable compositions are typically in the form of sterile solutions or suspensions, which include the active ingredient in a parenterally-acceptable diluent. Among these are sterile water, dextrose 5% in water (D5W), Ringer's solution and isotonic saline, as well as mixtures thereof. Cosolvents such as ethanol, propylene glycol or polyethylene glycols may also be used. Sterile, injectable oil is occasionally employed as a solvent or suspending medium in intramuscular preparations. A representative example is peanut oil. In addition, fatty acids such as oleic acid, preservatives, buffers and local anesthetics find use in the preparation of intramuscular injectables.

The active ingredient may also be administered rectally or intravaginally as suppositories. These can be prepared by mixing the drug with a suitable non-irritating excipient which is solid at ordinary room temperature but molten at normal or elevated body temperature. Examples of such materials include cocoa butter and polyethylene glycols.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

The entire disclosures of all applications, patents and publications, cited herein and of corresponding U.S. Provisional Application Ser. No. 60/633,430, filed Dec. 7, 2004, are incorporated by reference herein in their entirety.

From the foregoing description, one skilled in the art can easily ascertain the essential characteristics of this invention and, without departing from the spirit and scope thereof, can make various changes and modifications of the invention to adapt it to various usages and conditions.

What we claim:

1. A method of preventing headaches in a subject in need thereof, comprising,
administering to said subject a beta-adrenergic receptor antagonist and cyclooxygenase-3 inhibitor in amounts which are effective to prevent said headaches.

2. A method of claim 1, wherein the headache is a migraine headache, tension headache, or chronic daily headache.

3. A method of claim 1, wherein the frequency, severity, and duration of said headache is reduced.

4. A method of claim 1, wherein the beta-adrenergic receptor antagonist is bisoprolol, metoprolol, nadolol, nebivolol, timolol or propranolol.

5. A method of claim 1, wherein the cyclooxygenase-3 inhibitor is meloxicam, nabumetone, naproxen, oxaprozin, and piroxicam.

6. A method of claim 1, wherein the cyclooxygenase-3 inhibitor is selective for cyclooxygenase-3.

7. A method of claim 1, comprising consisting essentially of administering a beta-adrenergic receptor antagonist and a cyclooxygenase-3 inhibitor.

8. A method of claim 1, wherein said inhibitor does not possess substantial cyclooxygenase-2 inhibitory activity

9. A method of claim 1, wherein said cyclooxygenase-3 inhibitor is also a cyclooxygenase-1 inhibitor.

10. A method of claim 1, wherein said cyclooxygenase-3 inhibitor is a more potent inhibitor for cyclooxygenase-3 than for cyclooxygenase-1.

11. A method of claim 1, wherein said beta-adrenergic receptor antagonist and cyclooxygenase-3 inhibitor are administered sequentially.

12. A method of claim 1, wherein said beta-adrenergic receptor antagonist and cyclooxygenase-3 inhibitor are administered simultaneously.

13. A method of claim 1, wherein said beta-adrenergic receptor antagonist and cyclooxygenase-3 inhibitor are administered simultaneously and once every 24-hours.

14. A method of claim 13, wherein said wherein said beta-adrenergic receptor antagonist and cyclooxygenase-3 inhibitor are administered at bedtime.

15. A method of claim 1, wherein said amounts are synergistic.

16. A method of claim 1, wherein said beta-adrenergic receptor antagonist lacks intrinsic sympathomimetic activity.

17. A method of claim 1, wherein said beta-adrenergic receptor antagonist and cyclooxygenase-3 inhibitor are administered in the same composition.

18. A method of claim 1, wherein said beta-adrenergic antagonist is a beta-1 selective antagonist.

19. A method of preventing migraine headaches in a subject in need thereof, comprising,
administering to said subject a beta-adrenergic receptor antagonist and cyclooxygenase-3 selective in amounts which are effective to prevent said headaches.

20. A method of making a preparation for preventing headaches, comprising,
determining that an agent is a cyclooxygenase-3 inhibitor, and
preparing a preparation that comprises said cyclooxygenase-3 inhibitor.

21. A method of claim 20, wherein a beta-adrenergic antagonist is present in said composition or in a separate dosage unit.

22. A method of claim 20, further comprising administering said preparation to a subject in amounts which are effective to prevent said headaches in said subject.

23. A pharmaceutical composition comprising a cyclooxygenase-3 inhibitor and a beta-adrenergic receptor antagonist.
